(19) United States
(12) Patent Application Publication
(10) Pub. No.: US 2024/0009104 A1
(43) Pub. Date: Jan. 11, 2024

(54) PROCESS FOR EXTRACTION AND HEMI-SYNTHESIS OF PYRANOANTHOCYANINS AND SKINCARE COSMETIC FORMULATIONS CONTAINING THEM

(71) Applicants: UNIVERSIDADE DO PORTO, Porto (PT); REQUIMTE - REDE DE QUÍMICA E TECNOLOGIA, Porto (PT)

(72) Inventors: Iva Luzia REIS FERNANDES, Porto (PT); Joana Alexandra DA SILVA OLIVEIRA PINTO DA SILVA, Porto (PT); Nuno Filipe DA CRUZ BATISTA MATEUS, Porto (PT); Victor Armando PEREIRA DE FREITAS, Porto (PT); Helder José COUTO OLIVEIRA, Porto (PT); Paula Alexandra CARNEIRO ARAUJO, Porto (PT); Patricia RODRIGUES CORREIA, Porto (PT); Ana Rita MARTINS PEREIRA, Porto (PT)

(21) Appl. No.: 18/264,509

(22) PCT Filed: Feb. 9, 2022
(86) PCT No.: PCT/IB2022/051164

§ 371 Date: Aug. 7, 2023

(30) Foreign Application Priority Data
Feb. 9, 2021 (PT) 117058

(51) Int. Cl. A61K 8/49; A61Q 17/04; A61Q 19/08

(52) U.S. Cl. CPC A61K 8/498; A61Q 17/04; A61Q 19/08

(57) ABSTRACT

A process of extraction of anthocyanins from different foodstuffs or by-products, and their conversion into pyranoanthocyanins compounds, which can be used as pigments having new colors and improved stability to be incorporated into skincare formulations in particular anti-ageing and sun-protection formulations. Cosmetic formulations having the pigments with improved properties of UV protection and moisturizing, anti-dark spots, anti-wrinkles and wound-healing, which can be associated with interesting colors ranging from red to blue depending on the selected pigments in the composition and in the formula composition. The present invention is in the area of organic chemistry, biochemistry, pharmacology and cosmetics for skincare.

### TECHNICAL DOMAIN

The present invention is related to the process of extraction of anthocyanins from different foodstuffs or by-products, and their conversion into pyranoanthocyanins compounds, which can be used as pigments having new colours and improved stability to be incorporated into skincare formulations in particular anti-ageing and sun-protection formulations.

Cosmetic formulations comprising said pigments according to the invention present improved properties of UV protection and moisturizing, anti-dark spots, anti-wrinkles, and wound-healing, which can be associated with interesting colours ranging from red to blue depending on the selected pigments in the composition.

The present invention is in the area of organic chemistry, biochemistry, pharmacology and cosmetics for skincare.

### PRIOR ART

Chronical sun exposure, the main source of ultraviolet radiation (UVR), is the predominant cause of oxidative stressin the skin and one of the most extensively studied factors contributing for the aging process. The set of changes that the skin undergoes as a result of the damage induced by solar UVR is termed photoaging. The features of photodamaged skin commonly overshadow those of intrinsic aging, as they tend to appear earlier, are considerably more pronounced and estimated to account for up to 90% of visible skin aging.

The more prominent structural changes occur within the dermis, the layer that gives the skin its strength, elasticity and firmness, mainly consisting of an extracellular matrix (ECM) made of collagen and elastin; and to a lesser extent, proteoglycans and glycosaminoglycans (hyaluronic acid backbone) which provide hydration to the skin due to their great water retention capacity.

Matrix metalloproteinases (MMPs), a family of ubiquitous endopeptidases, play a vital role in the molecular mechanisms underlying ECM protein degradation as their expression has shown to be considerably elevated during the aging process, particularly in photodamaged skin.

Briefly, UVR incidence induces ROS production within keratinocytes and dermal fibroblasts, promoting the expression of several MMPs involved in collagen degradation, including MMP-1 (interstitial collagenase) resulting in accumulation of fragmented and irregularly distributed collagen fibrils and compromising the structural integrity of the dermal ECM.

The elastic fibre system also suffers significant structural changes as a result of MMPs upregulated expression. MMP-12 (macrophage elastase) plays a crucial role in elastin degradation and development of solar elastosis, a hallmark of photoaging, that consists of an abnormal accumulation of coarsen, disorganized and non-functional elastic fibres. Overall, these mechanisms typically emerge in the form of deepwrinkling, laxity, severe atrophy, and leathery appearance.

Another relevant feature and cosmetic concern associated with photoaging is the manifestation of skin hyperpigmentation. Melanin is synthesized in epidermal melanocytes by tyrosinase, within specialized organelles termed melanosomes, and subsequently transferred to neighbouring keratinocytes where it accumulates and shields the nuclear DNA by absorbing and scattering UVR.

Anthocyanins are water-soluble pigments that, depending on their pH may appear red, purple, blue or black. Food plants rich in anthocyanins include the blueberry, raspberry, black rice, and black soybean, among many others that are red, blue, purple, or black. Some of the colours of autumn leaves are derived from anthocyanins. Pyranoanthocyanins, a specific type of anthocyanins-derivatives are the major polyphenolic pigments formed in red wines during their ageing and maturation are thought to contribute to the orange hues observed in those wines during ageing.

Anthocyanins can be converted into pyranoanthocyanins chemically by cyclic addition onto carbon 4 and the hydroxyl group at the carbon 5 position of the anthocyanin, yielding a fourth ring that is responsible for the higher stability to hydration of these compounds when compared to the original anthocyanins.

Formulae A presents the general formula of pyranoanthocyanin pigments in flavylium cation form,

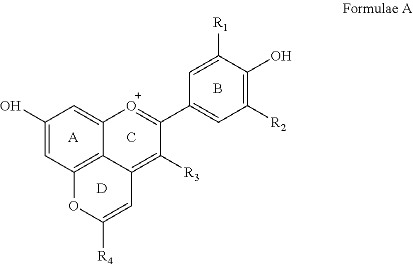

where typically:
R₁ is H, OH, OCH₃
R₂ is H, OH, OCH₃
R₃ is H, OH, O-sugar or acylated sugar,
R₄ is H; COOH; CH₃; COCH₃; O; cinnamyl group hydroxylated or/and methoxylated

The diverse colours presented by pyranoanthocyanins pigments and their higher colour stability, namely their colour stability at a wide pH range are important features indicating a putative application of these compounds in cosmetic products. Also, the more hydrophobic nature of these compounds makes their inclusion in lipophilic formulation much easier.

Over the years, several families of pyranoanthocyanins have been described in the literature including A and B-type vitisins, methylpyranoanthocyanins, oxovitisins, acetylpyranoanthocyanins, pyranoanthocyanin-phenolics, pyranoanthocyanin-flavanols, A and B-type portisins, pyranoanthocyanin dimers and pyranoanthocyanin-butadienilydene-phenolics, as shown in FIG. 1.

A-type vitisins or carboxypyranoanthocyanins result from the reaction between anthocyanins and pyruvic acid or oxaloaceticacid.

B-type pyranoanthocyanins can be formed from the reaction of anthocyanins with acetaldehyde.

Methylpyranoanthocyanins are yellowish pyranoanthocyanins and their formation was proposed to arise from the reaction of anthocyanins with acetone or acetoacetic acid. Pyranoanthocyanin-catechins and pyranoanthocyanin-catechols are orange pigments derived from the reaction of anthocyanins with vinyl-catechin or with vinyl-catechol, respectively.

Vinylpyranoanthocyanin-phenolics, commonly known as A and B-type portisins are bluish pyranoanthocyanin pigments. Their formation can derive from the reaction of carboxypyranoanthocyanins with (+)-catechin in the presence of acetaldehyde or with hydroxycinnamic acids, respectively.

Some of these reactions result from the metabolites produced by yeasts during wine fermentation (FIG. 2), whilst others involve other phenolic compounds present in wine. Amino based pyranoanthocyanins are naturally inspired but are synthetic analogues.

The inclusion of anthocyanin extracts to cosmetic formulations is known in the art to improve its UV absorption ability and solar protection factor.

Formulations comprising anthocyanin extracts usually present pH and density stability, pink colour and creamy aspect, although indirect light and stove conditions resulted in some extent of colour change indicating a degree of low stability.

Other documents disclose the photoprotective effects of strawberry-based formulations enriched with Coenzyme Q10 (CoQ10) when tested in UV exposed human dermal fibroblasts. Cells treated with the formulations having higher concentrations of strawberry extract, comprising pelargonidin and cyanidin glycosides as the most representative anthocyanin components, show ability to protect the cells from UV radiation harmful effects, restoring the cellular viability to similar values of those observed in non-irradiated cells.

Furthermore, the application of anthocyanins has also been explored as a skin whitening agent to reduce hyperpigmentation, a common evidence of photoaged skin. Tyrosinase is a major rate-limiting enzyme of melanin biosynthesis. For this reason, tyrosinase inhibitors have been extensively explored for the treatment of dermatological issues, such as solar lentigines and melasma. In this sense, anthocyanins from the Hibiscus syriacus L. shown capacity to decrease melanin production both in α-MSH stimulated B16F10 murine melanocytes and zebrafish larvae.

Stability of anthocyanins within the final formulation is essential to preserve the desired pharmacological effects. Complexation with di- or trivalent metal ions and co-pigmentation with phenolic acids and other flavonoids are some of the existing stabilization strategies that could be extensively explored in the context of development of dermatological formulations to secure the structural integrity, photoprotective and antiaging activities, as well as the natural appealing colour of anthocyanins.

Cosmetic formulations comprising natural compounds for antiaging applications are known in the art. Examples are disclosed in WO2007135132A1, EP2979684A1 and EP3375433A1.

However, none of these documents disclose anti-aging formulations comprising one or more pyranoanthocyanins, in particular pyranoanthocyanins obtained by conversion of anthocyanins from different foodstuffs or their by-products.

Further, other skincare formulations such as sunscreens or formulations for protecting the skin from sunlight, in particular natural cosmetics with UV protection typically make use of physical filters, which result in unaesthetic formulas with opacity and low fluidity due mainly to the low solubility of zinc oxide and titanium dioxide in water and oil compositions providing a white layer over the skin after application.

In consequence, the effective use of said compositions is heavily compromised. Moreover, they commonly restrict the use of current formulations to children.

One of the approaches to overcome this issue consists in adding organic UV filters to reduce their opacity and increase their fluidity. Although this is an effective approach, organic sunscreens like oxybenzone, octinoxate, octisalate, octocrylene, homosalate, avobenzone, aminobenzoic acid, and trolamine salicylate can present cellular toxicity to humans and marine environment and therefore, said formulations are often not recognized as safe and effective (GRASE). Moreover, recent trends prohibit the use of this type of products and formulations in fragile marine environment to protect the resident fauna and flora.

To overcome the cited prior art problems, the present invention proposes cosmetic formulations comprising pyranoanthocyanin extracts, since it was observed that they can replace the need of any of the above mention ingredients with the additional advantage of being more efficient and avoid the addition of synthetic undesirable compounds.

These extracts are obtained by the method of the invention comprising the extraction of anthocyanins from different foodstuffs or their by-products and further converting them into the desired pyranoanthocyanin conferring specific properties to the formulations thereof.

### DESCRIPTION OF THE FIGURES

FIG. 1 Shows the structure of several pyranoanthocyanin compounds according to the invention, having formula I to X, which can be used as pigments.

FIG. 2 Representative spectra of the absorbance of different pyranoanthocyanins at 0.2 mg/mL in ethanol.

FIG. 3 Representative spectra of the absorbance of carboxypyranocyanidin-3-glucoside and methylpyranocyanidin-3-glucoside at different concentrations of zinc oxide.

FIG. 4 shows the inhibitory activity of pyranoanthocyanins against tyrosinase, wherein:

FIG. 4a. shows, the inhibitory activity of carboxypyranocyanidin-3-glucoside and methylpyranocyanidin-3-glucoside, carboxypyranomalvidin-3-glucoside and methylpyranomalvidin-3-glucoside and Kojic acid (50 μM), against tyrosinase,

FIG. 4b. shows the inhibitory activity, at different concentration, of carboxypyranocyanidin-3-glucoside and methylpyranocyanidin-3-glucoside against tyrosinase.

FIG. 5 shows the inhibitory activity of carboxypyranocyanidin-3-glucoside and methylpyranocyanidin-3-glucoside, carboxypyranomalvidin-3-glucoside and methylpyranomalvidin-3-glucoside (50 μM) against hyaluronidase.

FIG. 6 shows the inhibitory activity of pyranoanthocyanins against elastase, wherein:

FIG. 6a shows the inhibitory activity of carboxypyranocyanidin-3-glucoside and methylpyranocyanidin-3glucoside (50 μM), against elastase,

FIG. 6b shows the inhibitory activity of carboxypyranomalvidin-3-glucoside and methylpyranomalvidin-3glucoside (50 μM) against elastase.

FIG. 7 shows the inhibitory activity of pyranoanthocyanins (50 μM) against collagenase, wherein:

FIG. 7a. shows the inhibitory activity of carboxypyranocyanidin-3-glucoside and methylpyranocyanidin-3-glucoside, against collagenase,

FIG. 7b. shows the inhibitory activity of carboxypyranomalvidin-3-glucoside and methylpyranomalvidin-3-glucoside (50 μM) against collagenase.

FIG. 8 Cytotoxicity activity of carboxypyranocyanidin-3-glucoside, methylpyranocyanidin-3-glucoside and carboxypyranomalvidin-3-glucoside, evaluated by MTT assay, wherein:

FIG. 8a Primary Epidermal Keratinocytes; Normal, Human, Adult (HEKa) (ATCC® PCS-200-011™),

FIG. 8b spontaneously transformed aneuploid immortal keratinocyte cell line from adult human skin (HaCat),

FIG. 8c normal human foreskin fibroblasts (HFF-1).

FIG. 9 Effect of methylpyranocyanidin-3-glucoside and carboxypyranomalvidin-3-glucoside, at 50 μM, on ROS production after 24 h in HFF-1 cells, using fluorescent the dye DCFDA. Hydrogen peroxide was used as a positive control.

FIG. 10 Effect of pyranoanthocyanins, at 50 μM, on superoxide dismutase (SOD) activity, wherein:

FIG. 10a Evaluated in keratinocytes cell line,

FIG. 10b Evaluated in fibroblasts cell line.

FIG. 11 Biofilm inhibition (%) in the presence of pyranoanthocyanins, wherein:

Biofilms of a) P. aeruginosa (ATCC 27853) and b) S. aureus (ATCC 25923) were formed in the presence of mixture B, comprising carboxypyranocyanidin-3-glucoside, methylpyranocyanidin-3-glucoside and carboxypyranomalvidin-3glucoside at three different concentrations, MIC, ½×MIC, and ¼×MIC. Control biofilms were grown in absence of the compound. Two independent experiments were performed in triplicate.

Error bars represent SD.

FIG. 12 Transport efficiency of carboxypyranocyanidin-3-glucoside through HeKa cells (apical-basolateral). Results are presented as transport efficiency (%) (mean±SEM). Transport efficiency percentages were calculated based on (compound concentrations at the basolateral side overtime)/(compound concentrations at the apical side at the zero hours)×100.

FIG. 13 Time needed for HaCat cells to recover 50% from the injury caused at wound in the presence of DMEM-F12 medium (Control) or in the presence of carboxypyranocyanidin-3-glucoside. Each value represents the mean±SEM (n=4-5).

### DESCRIPTION

The present invention is related to the process of extraction of anthocyanins from different foodstuffs or by-products, and their conversion into pyranoanthocyanins compounds, which can be used as pigments having new colours and improved stability to be incorporated into skincare formulations in particular anti-ageing and sun-protection formulations.

These reactions involve industrial foodstuffs or by-products as anthocyanin source to obtain anthocyanin derivatives having similar structural diversity as the natural ones, hydroxylation and methoxylation patterns in ring B and sugar moieties with different degree of acylation but with different, substituents in ring D as shown in FIG. 3.

### 1. Extraction of Anthocyanins

Anthocyanins can be extracted from different sources (fruits, agro-food by-products). In the present invention, anthocyanins are preferably extracted from foodstuff or their by-products such as from winemaking industry. Another adequate source of anthocyanins is from redberries, blackberries, blueberries.

The anthocyanins present in said products can be subjected to an extraction step by using acidified hydroalcoholic solvents and purified by column chromatography with C18 reverse-phase gel by low-pressure column chromatography to obtain anthocyanins.

Anthocyanins can be concentrated by nanofiltration technologies and used in aqueous solution for the hemi-synthesis of pyranoanthocyanins or reduce to a fine powder by spray drying or freeze drying before the hemi-synthesis step.

The resulting fractions can be analysed by HPLC-DAD/MS for identification and quantification of extracted anthocyanins.

Several structurally different anthocyanins can be detected depending on their source, although only anthocyanins non-glycosylated on position 5-O are further used for the hemi-synthesis step.

It is also important to notice that the whole extract or one purified compound can be used for the synthesis of pyranoanthocyanins.

### 2. Hemi-Synthesis of Pyranoanthocyanins

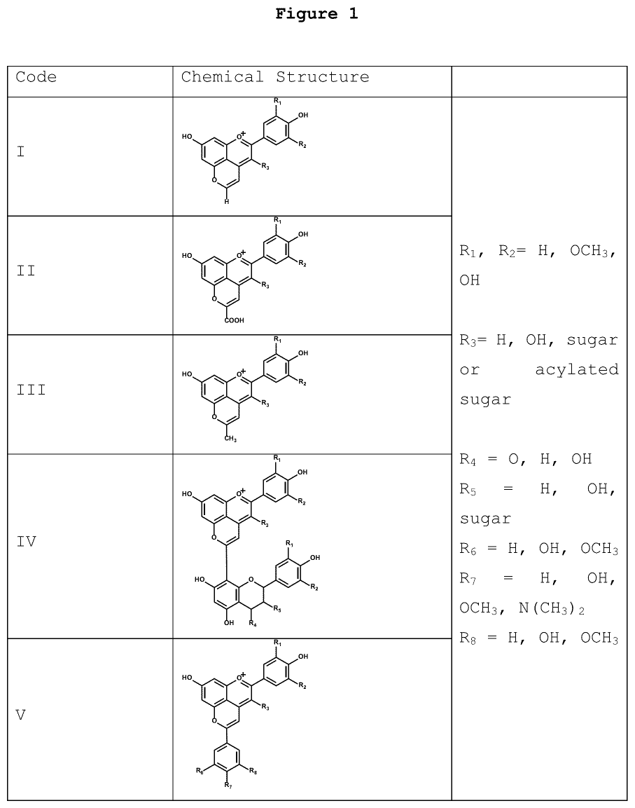

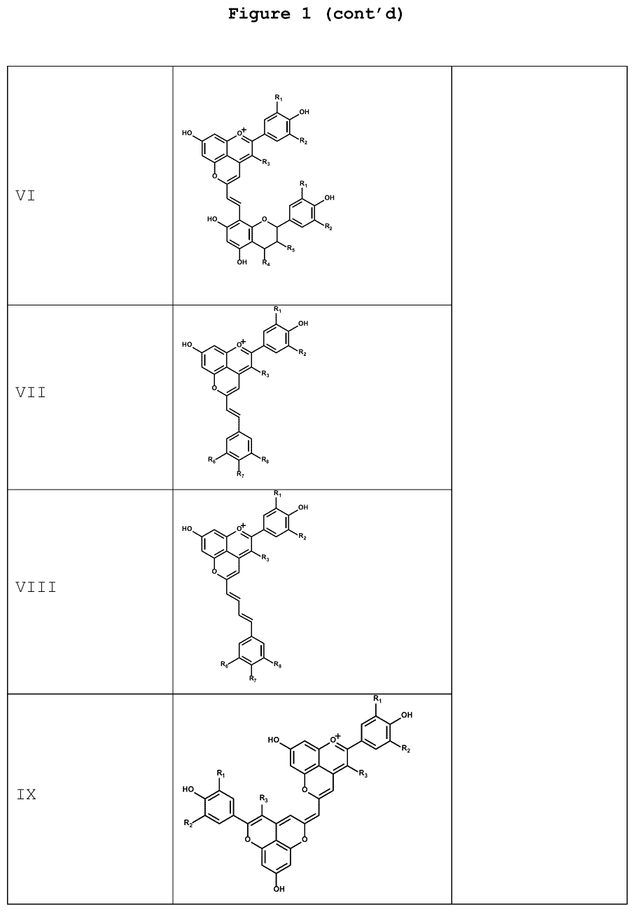

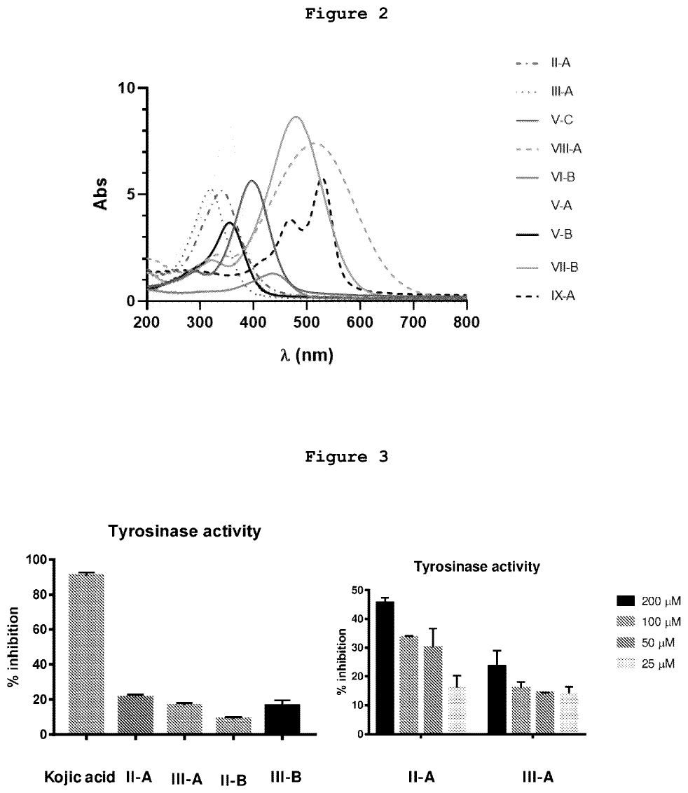

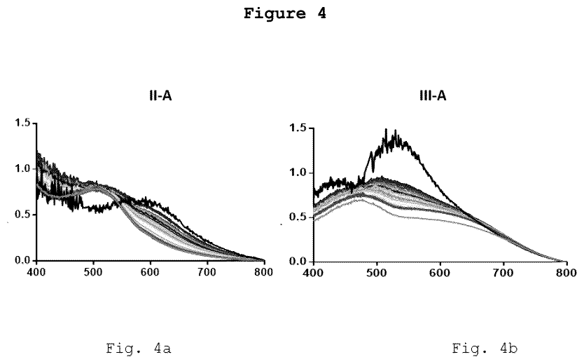

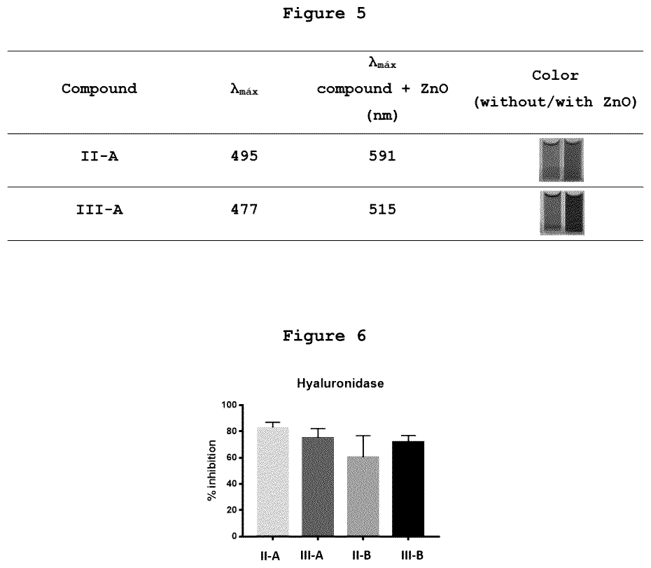

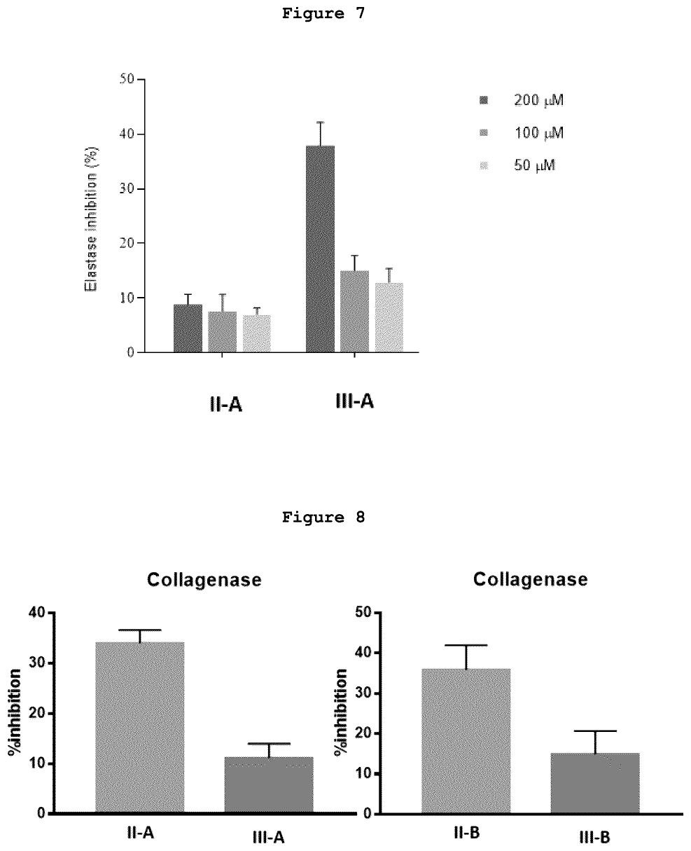

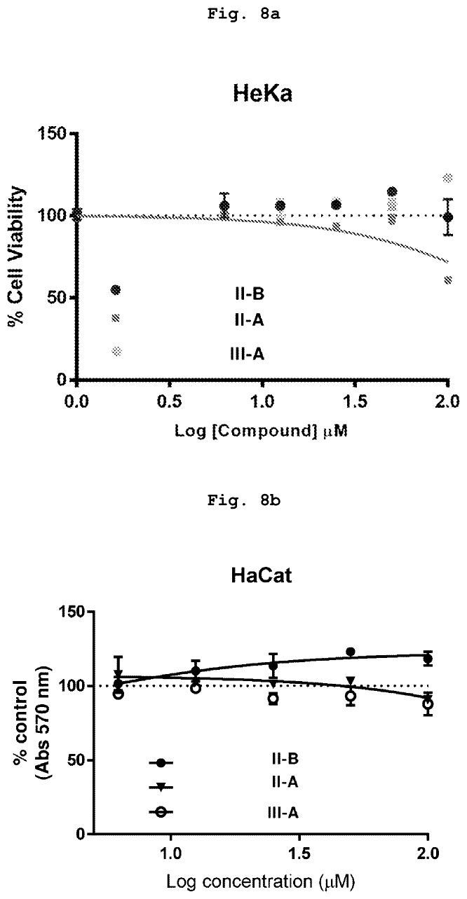

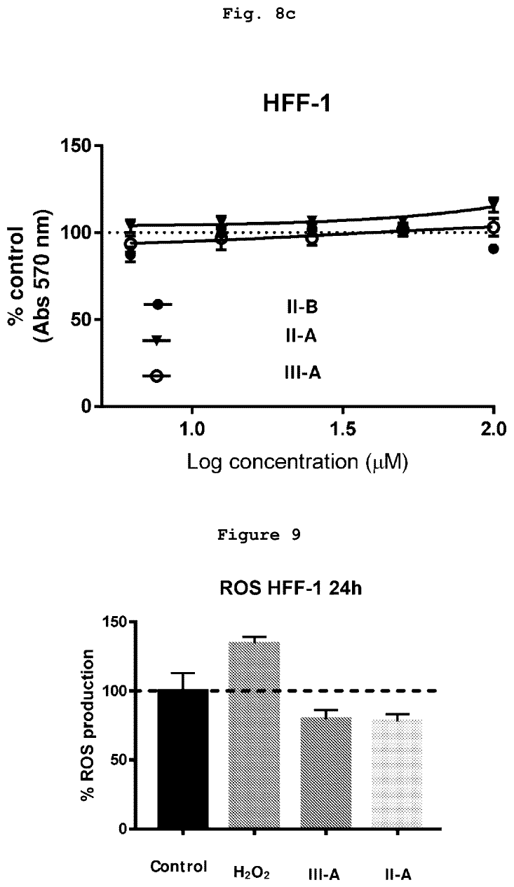

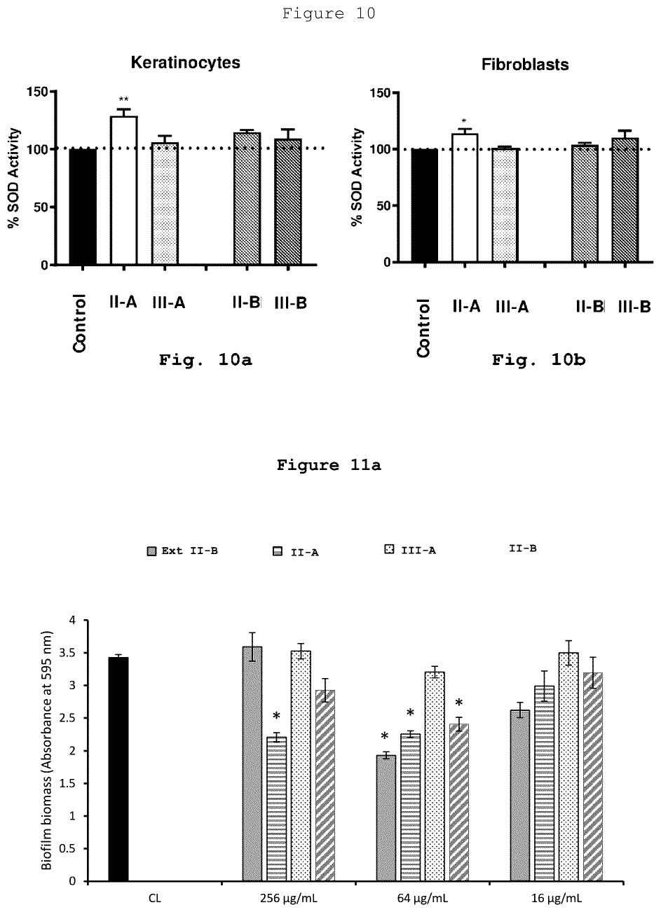

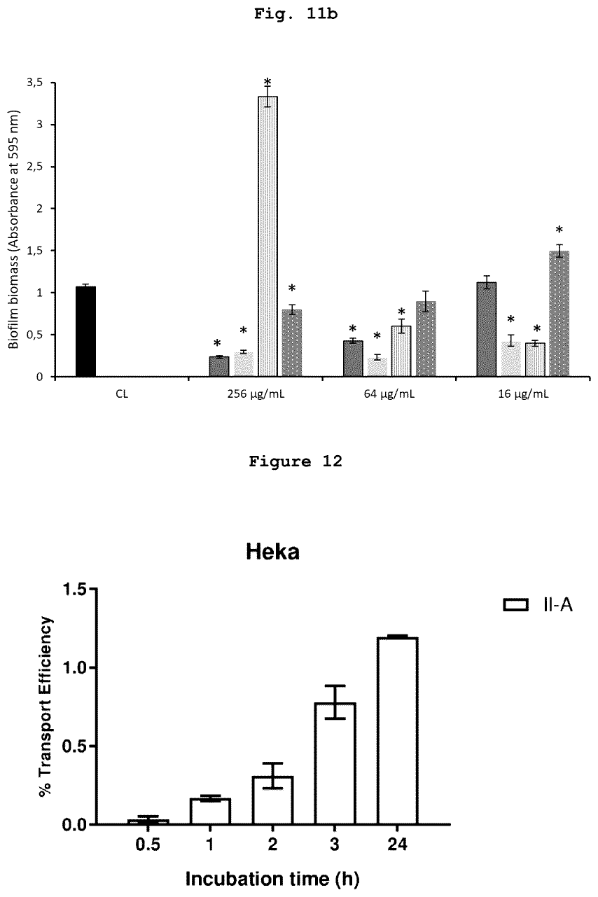

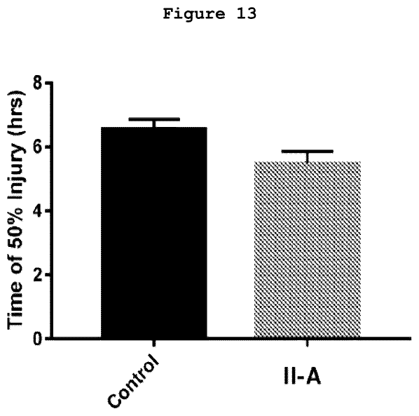

Anthocyanins extracted as above-mentioned can be converted into pyranoanthocyanins through chemical transformation. Non-glycosylated on position 5-O anthocyanins are subjected to a hemi-synthesis process by reaction with a precursor (R—C═CH₂, R being the substituent that characterizes the family of compounds from I-IX) that yields to the formation of a fourth ring in the pyranoanthocyanin compound. The resulting extracts are then purified for example by reverse-phase silica C-18 gel column chromatography with each pyranoanthocyanin family (see FIG. 1) being eluted with a specific % of alcoholic solution such as water/ethanol (acidified with HCl). Extracts without any purification can also be included in cosmetic formulations.

### Food Grade Synthesis

Hydroxycinnamic acids, pyruvic acid (PA), acetaldehyde and acetoacetic acid food grade, commercially available can be used to produce pyranoanthocyanins.

Apart from the chemical process some of the above-mentioned compounds can be produced by reaction of hydroxycinnamic acids (p-coumaric, caffeic, ferulic, or sinapic) or through their decarboxylation products (4-vinylphenols) with anthocyanins, giving rise to pinotins.

Besides, natural fruit extracts (pear, apple, among others) could also be a source of hydroxycinnamic acids. For example, pear extract is rich in ferulic acid and can be used to produce anthocyanin-vinylguaiacol type pyranoanthocyanins.

### Yeast Mediated

Red fruits extracts of anthocyanins were converted into carboxy-pyranoanthocyanins containing extracts from the reaction of anthocyanins with PA produced by yeasts. PA is produced during the fermentation or respiration phase of Saccharomyces and non-Saccharomyces yeasts at 25-30° C. and pH 4.0-4.5 using a medium containing sugar and a nitrogen source. The detection and quantification of pyruvic acid (PA) in model solutions was performed in a Thermo® Scientific HPLC by injecting 20 μL of each sample on a 300×7.8 mm i.d. anion exclusion column (Grace Davison, Columbia, SC, USA) at 50° C.

The detection was carried out at 214 nm and the solvent use d was 2.5 mM of H₂SO₄ at 0.35 mL/min for 30 min. Calibration curves were obtained using PA standards.

At the end of fermentation, chemical analyses were performed by HPLC to quantify the produced carboxy-pyranoanthocyanin.

At the end of the fermentation, the medium is nutritionally depleted and the yeast starts to reuse part of the excreted PA. At that time the synthesis of type B vitisins begins since the production of acetaldehyde is increased.

Methylpyranoanthocyanins group derived from the reaction between anthocyanins and yeast metabolites acetoacetic acid was also obtained in the same fermentation conditions.

Vinylphenols essential to form pinotins were formed via enzymatic decarboxylation of p-coumaric, caffeic, ferulic, and sinapic acids by Saccharomyces and non-Saccharomyces yeasts during fermentation.

### 3. Pyranoanthocyanins

The resulting pyranoanthocyanins can be characterized by the formula I to VIII of FIG. 1, according to the type of molecule or group linked to the carbon C10 of the fourth ring.

These anthocyanin-derived compounds display different physical-chemical properties from their anthocyanin precursors, especially chromatic features. In general, pyranoanthocyanins have a maximum absorption wavelength hypsochromically/bathochromically shifted from −520 nm to 478-510 nm/570/670 nm, which results in a yellow-orange/blue colour of these pigments compared to a red-purple hue of genuine anthocyanins.

### 4. Cosmetic Formulations

The pH equilibrium forms of pyranoanthocyanins confer to the final formula of a cream/textile a range of different colours. This feature is controlled and fit in the range of pH values of 4-6. Otherwise, the structure of the bioactive or the target of the invention (skin care application) will be impaired.

Within that range of the above pH values, it is possible to increase the intensity of the final coloration and also to change it by the addition of co-pigments such as other phenolic compounds or metals being the complexation with zinc oxide an adequate example. Further, the ability of these compounds to absorb light at the UV range, permit the reduction of the physical filters normally present in natural sunscreens.

The final formulations can include the pyranoanthocyanins at a maximum of 0.5%, with or without up to 10% zinc oxide in a base formulation.

### Determination of Solar Protection Factor

Sample preparation 1.0 mg of all samples was weighed, transferred to a 5 mL volumetric flask, diluted to volume with ethanol, followed by ultra-sonication for 5 min. The absorption spectra of samples in solution were obtained in the range of 290 to 450 nm using 1 cm quartz cell, and ethanol as a blank. The absorption data were obtained in the range of 290 to 320, every 5 nm, and 3 determinations were made at each point, followed by the application of Mansur equation.

$${SPF}_{spectrophootometric} = CF \times {\sum }_{290}^{320} EE ( λ ) \times I ( λ ) \times Abs ( λ )$$

TABLE 1
Normalized product function used in the calculationof SPF.

| | EE × I |
|---|---|
| λ(nm) | (normalized) |
| 290 | 0.015 |
| 295 | 0.0817 |
| 300 | 0.2874 |
| 305 | 0.3278 |
| 310 | 0.1864 |
| 315 | 0.0839 |
| 320 | 0.018 |
| Total | 1 |

EE—erythemal effect spectrum;
I—solar intensity spectrum

Where CF (correction factor) is 10, EE (A) is erythmogenic effect of radiation with wavelength λ, Abs is Spectrophotometric absorbance values at wavelength λ. The values of EE (λ)×1 (λ) are constant and can be refer to Table 1. The obtained absorbance values are multiplied with EE (λ)×1 (λ) and then their summation is taken and multiplied with correction factor to obtain the SPF values.

### 5. Evaluation of Biological Activity of Pyranoanthocyanins

The biological activity of pyranoanthocyanins obtained according to the above-mentioned method can be evaluated by conventional assays, in particular the following assays:

### 5.1 Superoxide Dismutase (SOD) Activity

The evaluation of the activity of superoxide dismutase can be evaluated after treating the keratinocytes or fibroblasts with different concentrations of each pyranoanthocyanin. Superoxide dismutases (SODs) are a group of metalloenzymes that form the front line of defense against reactive oxygen species (ROS)-mediated injury.

### 5.2 Collagenase Activity

About 80% of the dry weight of the dermis is made up of collagens, of which the predominant types are types I and III[8]. Collagen fibres have a pivotal role in the integrity of skin and wound healing.

Among the different types of MMPs, collagenases (MMP-1) playa pivotal role on the degradation of the main support blocks of skin, collagen.

### 5.3 Elastase Activity

Elastic fibers account for about 5% of the dry weight of the dermis and consist of complex structures of polymerized elastin and microfibrils. Elastic fibres are essential building blocks that confers elasticity to the skin.

Elastases (MMP-12) play a pivotal role on the degradation of elastin. The excessive degradation of elastin will provoke serious damage on the skin elasticity.

### 5.4 Tyrosinase Activity

In normal conditions, melanin is essential for keratinocyte protection, however, the overproduction of this pigment can lead to hyperpigmentation but also freckles, wrinkle formation, melisma, and is related to the formation of melanomas mainly due to the alteration of skin homeostatic balance. Such phenomenon is associated with an overexpression of tyrosinase, which is associated with the continuous exposure to UV radiation and the other factors promoters of skin aging. One of the strategies to overcome the effects of UV radiation (and all the other external aggressions that result in an imbalance of skin homeostasis), is by reducing the activity of the key enzymes involved in skin aging, such as tyrosinase.

### 5.5 Hyaluronidase Activity

Hyaluronidases belong to the class of glycosidases and have the main role of degrading hyaluronic acid. These enzymes can be found widespread in different organs of the body, including skin. As hyaluronic acid is essential as a lubricant for soft connective tissue but also important for keratinocytes differentiation and wound healing, thus higher activities of hyaluronidase will result in an impaired function of hyaluronic acid. In fact, long-term exposure to UV has been shown to reduce hyaluronic acid in the dermis, due to lower hyaluronan synthases expression and progressive inactivation of fibroblasts, which results in a higher activity of hyaluronidases.

### 5.6. Wound-Healing Evaluation

Evaluation of wound healing ability of each formulation can be performed by using a microelectrode-based biosensor device, referred to as Electric Cell-Substrate Impedance Sensing (ECIS). The ECIS device monitors the impedance of small gold-coated electrodes used as support for cells in culture and can be used to detect subtle changes in the cell-matrix interaction. Wound-healing assays in ECIS are performed by applying a high electrical current to a confluent cell monolayer, creating an empty space on the surface of the electrode. The healing process is then monitored continuously, measuring impedance over time. These measures reflect the migration and proliferation of cells from the microelectrode surroundings to its surface.

### 5.7 Cell Culture Conditions

For this purpose, immortalized human foreskin fibroblasts HFF-1 were grown as monolayers from passage number 9-18 and maintained at 37° C. in an atmosphere of 5% CO₂. For routine maintenance, the cells were cultured in 22.1 cm² plates as monolayers and maintained in Dulbecco's Modified Eagle Medium (DMEM, from Cell Lines Service), supplemented with 10% fetal bovine serum (FBS, from CLS) and 1% of antibiotic/antimycotic solution (100 units/mL of penicillin, 10 mg/mL of streptomycin and 0.25 mg/mL of amphotericin B from Sigma-Aldrich, St. Louis, MO, United States). The medium was replaced every two days and the cells were harvested every two weeks.

### 5.8 Cytotoxicity to Human Fibroblasts and Keratinocytes

The cytotoxicity of the anthocyanins derivatives to HFF-1, Heka, and HaCat cells was evaluated using the standard MTT assay. Briefly, cells were seeded at a density of 5×10⁴ cells or 1.5×10⁴ cells/well onto 96-well plates and incubated at 37° C. in a 5% CO₂ atmosphere. Cells were allowed to grow for 24 h, and serially diluted solutions of the different compounds (0.78-100 μM) were added to the wells. Then, cells were incubated for 48 h at 37° C., after which wells were washed once with phosphate buffered saline solution (PBS, Sigma-Aldrich, St. Louis, MO, United States), followed by addition of 0.45 mg/mL of MTT solution to each well. Crystals were allowed to form for 2 h. Reaction was stopped by rejecting the medium and addition of dimethylsulfoxide (DMSO, Sigma-Aldrich, St. Louis, MO, United States). Absorbance was read at 570 nm.

### 5.9 Reactive Oxygen Species Experiments

The reactive oxygen species (ROS) production in normal human foreskin fibroblast, HFF-1 cells was evaluated following the standard method. Briefly, cells were seed at a density of 5×10⁴ cells/well onto 96-well plates and allowed to reach confluency. At this point, cells were washed twice with phosphate buffered saline solution (PBS, Sigma-Aldrich, St. Louis, MO, United States) and the different compounds were added at a concentration of 50 μM with an incubation period of 24 h at 37° C. in an atmosphere of 5% CO₂. After that, cells were washed twice in Hank's Buffered Saline Solution (HBSS) and incubated with 100 μL of 50 μM DCFDA for 30 minutes in the same previous conditions. The cells were then washed twice with HBSS and incubated with fresh DMEM medium for 24 h. After this period, the fluorescence intensity (ex 485/em 520) was then registered.

### 5.10 Cell Culture Conditions for Enzymatic Studies

The cells were seeded at a density of 5×10⁴ cells/well onto 12-well plates and allowed to reach confluency. At this point, cells were washed twice with phosphate buffered saline solution (PBS, Sigma-Aldrich, St. Louis, MO, United States) and the different compounds were added at a concentration of 50 μM with an incubation period of 24 h at 37° C. in an atmosphere of 5% C02. The cells were then washed twice with PBS. After this, the cells were placed in ice and the lysis buffer was applied (1 tablet of Pierce^{TH} Phosphatase Inhibitor Minitablets, Thermo and 1 tablet of complete Mini, EDTA-free, Roche in 10 mL of
Milli Q® ultrapure water). Then the cells were removed with scrappers to different eppendorfs according to the treatments. The cells were then placed on ice for 20 minutes with stirring and centrifuged at 13000 g for 10 minutes at 4° C. The supernatant was recovered and stored at −80° C.

### 6. Protein Assays

It is essential to normalize the total protein content of the samples after each treatment. For the same concentration, the enzymatic activity is determined.

The quantification of total protein in each sample can be evaluated by the standard methods such as bicinchoninic acid (BCA). Briefly, 25 μL of each sample was used in triplicate and incubated with the working reagent and the quantificationwas done accordingly the manufacturer protocol.

### 7. Determination of Total Superoxide Dismutase (SOD) Activity Modulation by Pyranoanthocyanins

Superoxide dismutases (SODs) are a group of metalloenzymes that form the front line of defense against reactive oxygen species (ROS)-mediated injury. These proteins catalyse the dismutation of superoxide anion free radical (O₂⁻) into molecular oxygen and hydrogen peroxide (H₂O₂) and decrease O₂⁻ level which damages the cells at excessive concentration.

For the determination of SOD activity, two solutions were prepared for the reaction system. Solution A: 5 mL of Xanthine7.23 mM in NaOH 0.1M mixed with Nitroblue tetrazolium (NBT) 83.2 μM in Phosphate Buffer (50 mM, EDTA 0.1 mM pH 7.8) at a final volume of 50 mL so that the final concentrations of Xanthine and NBT are 72.3 μM and 83.2 μM, respectively. Solution B: Xanthine Oxidase 0.5 U/mL in EDTA 0.1 mM.

Briefly, 50 μL of the previously obtained samples were mixed with 200 μL of a solution A. After this, 50 μL of solution B were mixed for a final reaction volume of 300 μL. As blank solution B was substituted by phosphate buffer. The reduction of NBT by reactive oxygen species was observed by following the absorbance at 560 nm for 180 seconds. The activity of SOD in each sample was calculated accordingly to the following equation:

$$A c {t}_{SOD} ( U \cdot {mL}^{- 1} ) = \frac{1}{Abs \cdot {min}^{- 1} ( 560 nm ) \times 1 , 9061} - 0 , 6155.$$

The experiments were done in triplicates.

### 8. Enzymatic Assays

Enzymatic activity of pyranoanthocyanins can be evaluated by evaluation of collagenase, elastase, tyrosinase and hyaluronidase activity.

### 8.1 Collagenase Activity

The assays for the determination of the enzymatic activity of collagenase in the presence of the different compounds were performed as follows: a stock Clostridium histolyticum collagenase was dissolved in 100 mM of Phosphate Buffer pH 7.4, at a concentration of a 125 U.ml⁻¹. The substrate N-[3-(2-furyl)-acryloyl]-Leu-Gly-Pro-Ala (FALGPA) and the different compounds were dissolved in the same buffer at the concentrations of 6 mM and 200 μM, respectively. Then the compounds (75 μL) were incubated with the substrate (30 μL) for 10 minutes and then the enzyme (40 μL) was added to initiate the reaction, followed by 35 minutes at 324 nm and 37° C. The final volume of the reaction was adjusted to 300 μL for each sample. The final concentrations of each component were as follows: enzyme 16.6 U.ml⁻¹, substrate 600 μM and compounds 50 μM. Buffer instead of compounds was used as a control.

The inhibition was calculated using the following formula:

$$\% Inhibition = ( 1 - \frac{{B}_{0} - {B}_{35}}{{A}_{0} - {A}_{35}} ) \times 100$$

Where B₀ represents the initial absorption of the reaction in the presence of pigments after 30 minutes of incubation and B₃₀ the final absorption of the reaction. A₀ and A₃₀ represents the same conditions but in the absence of the compounds.

### 8.2 Elastase Activity

The assays for the determination of the enzymatic activity of elastase in the presence of the different compounds were performed as follows: pancreatic porcine elastase was dissolved in 100 mM of Phosphate Buffer pH 6.8, at a concentration of a 2.34 U.mL⁻¹. The substrate N-Suc-(Ala)-3-p-nitroanilido and the different compounds were dissolved in the same buffer at the concentrations of 6 mM and 200 μM, respectively. Then the compounds (75 μL) were incubated with the substrate (30 μL) for 10 minutes and then the enzyme (20 μL) was added to initiate the reaction, followed by 35 minutes at 405 nm and 37° C. The final volume of the reaction was adjusted to 300 μL for each sample. The final concentrations of each component were as follows: enzyme 0.156 U.mL⁻¹, substrate 600 μM and compounds 50 μM. Buffer instead of compounds was used as a control.

The inhibition was calculated using the following formula:

$$\% Inhibition = ( 1 - \frac{{B}_{0} - {B}_{35}}{{A}_{0} - {A}_{35}} ) \times 100$$

Where B₀ represents the initial absorption of the reaction in the presence of pigments after 35 minutes of incubation and B₃₅ the final absorption of the reaction. A₀ and A₃₅ represents the same conditions but in the absence of the compounds.

### 8.3 Tyrosinase Activity

Tyrosinase inhibitory activity was determined using mushroom tyrosinase (Sigma Aldrich, T3824-250 KU) and 3,4-Dihydroxy-L-phenylalanine (L-DOPA) as enzyme and substrate, respectively.

Both were dissolved in 20 mM phosphate buffer solution, pH 6.8. First, 20 μL from a freshly prepared stock solution of mushroom tyrosinase (270 U/mL) and 75 μL of test compounds stock solutions (200 μM, in 20 mM PBS, pH 6.8) were mixed and pre-incubated at 37° for 15 min. Then, 30 μL of 6 mM L-DOPA were added to the reaction mixture, followed by an incubation of 20 min at 37° C. During this reaction L-DOPA is converted to dopachrome (a precursor of melanin), visually detected by the change of color from colorless to orange/brown, resulting in an absorbance increase, recorded spectrophotometrically at 475 nm on a FlexStation 3 Multi-Mode Microplate Reader (Molecular Devices).

The inhibition rate was calculated as follows:

$$\% Tyrosinase inhibition = ( 1 - \frac{A - B}{C - D} ) \times 100$$

Where A and B represent the final and initial optical densities of the reaction in the presence of the tested compounds and C and D represent the final and initial optical densities of the reaction in their absence, respectively. Experiments were carried out in triplicate and repeated at least 3 times.

### 8.4 Hyaluronidase Activity

The assays for the determination of the enzymatic activity of hyaluronidase in the presence of the different compounds were performed as follows: bovine testes hyaluronidase was dissolved in 50 mM of Phosphate Buffer pH 7.4 50 mM NaCl at a concentration of 20 U.mL⁻¹. The substrate bovine vitreous humour hyaluronic acid was prepared in Phosphate buffer 300 mMpH 5.35 at a concentration of 1 mg. mL⁻¹. The compounds were prepared in 50 mM of Phosphate Buffer pH 7.4 50 mM NaCl at a concentration of 200 μM. The stop solution hexadecyltrimethylammonium bromide (CTAB) was prepared in Phosphate buffer 50 mM pH 3.75 at a concentration of 3 mg.mL⁻¹. The enzyme (30 μL), the buffer (52.5 μL) and the compounds were incubated for 10 minutes. Then the substrate was added followed by a 45 minutes incubation. The final concentrations of the different components were: enzyme 4 U.mL⁻¹, substrate 0.2 mg.mL⁻¹ and compounds 50 μM. After this time, the stop solution was added to interrupt the reaction. After 10 minutes the absorbance was measured at 400 nm.

### 9. Wound-Healing Assays-ECIS System

### 9.1 Cell Culture

An aneuploid immortal keratinocyte cell line from adult human skin, HaCat, was grown as a monolayer from passage number 35 to 55. For routine maintenance, cells were cultured in 25 cm² monolayer in DMEM-F12 or RPMI (Sigma, Madrid, Spain) medium supplemented with 10% heat-inactivated FBS and 1% antibiotic/antimycotic solution (100 units mL⁻¹ of penicillin, 100 μg mL⁻¹ of streptomycin and 0.25 μg mL⁻¹ of amphotericin B) at 37° C. in a humified atmosphere with 5% CO₂. Cells were harvested by trypsinization (0.25% (w/v) trypsin-EDTA₄Na) twice a week.

### 10. Electric Cell-Substrate Impedance Sensing (ECIS) Assay

A commercial ECIS system (Applied Biophysics, Troy, NY, USA) was employed to measure the impedance associated with the proliferation of human keratinocyte cells. The ECIS system can simultaneously measure cell resistance and capacitance during cell culture. 8W1E sensing chips consisting of eight separate wells which were 1 cm in height and 0.8 cm² in bottom area were used. One detecting electrode of 250 μm in diameter is deposited on the bottom of each well and connected in series to a 1 MΩ resistor, an AC signal generator and a large gold counter electrode. Since the AC signal amplitude is set to 1 V, it results in a current source with the amplitude of 1 μA. The in-phase voltage was proportional to the resistance and the out-of-phase voltage was proportional to the capacitive reactance.

### 11. Keratinocyte Monolayer Model Optimization Using ECIS

A pre-treatment of the electrodes was performed as described in Szulcek, R., H. J. Bogaard, and G. P. van Nieuw Amerongen, Electric Cell-substrate Impedance Sensing for the Quantification of Endothelial Proliferation, Barrier Function, and Motility. J. Vis. Exp., 2014. 28 (85): p. e51300.

8W1E PET electrodes were used as they are ideal for wound healing assays. They were first treated with 10 mM L-cysteine solution for 15 min, for cleaning and to improve well-to-well reproducibility, and signal-to-noise ratio wells were washed 3 times with sterile ultrapure water and incubated with 1% gelatine solution for 30 min. After washing, 500 μL of DMEM-F12 medium were added to each well to check resistance and capacitance basal values.

If the cleaning process was successful (basal resistance values around 2000Ω and capacitance around 5 nF), cells were inoculated at two different cellular densities (0.8×106 cells/mL) with DMEM-F12, in a final volume of 500 μL. Cells were allowed to attach to the electrodes outside the incubator for 30 min. After 24 hours in culture, the confluency and viability of the cell monolayer was confirmed by light microscopy and electrically by impedance measurements. Half or total medium was renewed 20 h after inoculation and cells were allowed to adapt for 4 hours. Thereafter, 250 μL of compound solutions were applied to each well at a final concentration of 20 μM and the arrays were incubated for 24 hours. A positive control with DMSO was performed to validate cytotoxicity assays. The electrical impedance of each well was measured every 2 min and up to 16 individual wells were followed successively.

Cytotoxicity was assessed by taking impedance measures and applying the mathematical model developed by Giaever and Keese, as described in Giaever, I. and C. R. Keese, Micromotion of mammalian cells measured electrically. Proceedings of the National Academy of Sciences of the United States of America, 1991. 88 (17): p. 7896-7900.

It can be used to calculate morphological parameters, as the barrier function of the cell layer, the spacing between the ventral side of the cell and the substratum, and the cell membrane capacitance.

### 12. ECIS Wound Assay

Wound-healing assays were performed after incubation of HaCatcells with compounds for 24 hours. Wounding was performed by applying an AC current of 2400 μA, 60 kHz for 60 s, killing the cells on the surface of the electrode which resulted in an abrupt drop in impedance to values like those of a cell-free electrode. Healing process was monitored continuously as cells migrated and proliferated onto the electrode.

### 13. Antimicrobial and Antibiofilm Assays

Bacterial strains and growth conditions:

Compounds/extracts were tested against the following bacterial strains: Pseudomonas aeruginosa ATCC 27853, Staphylococcus aureus ATCC 29213, Staphylococcus epidermidis ATCC 14990, Streptococcus pyogenes ATCC 19615, Micrococcus luteus ATCC 4698. Prior to each in vitro bioassay, fresh cultures were obtained for each strain using the appropriate medium and incubation conditions as follows. Staphylococcus spp. and P. aeruginosa were grown on Mueller-Hinton (MH) agar (Liofilchemsrl, Italy) for 24 h at 37° C., while M. luteus was grown in Tryptic Soy agar (TSA, Liofilchem srl, Italy) for 24 h at 30° C. and S. pyogenes on TSA with 5% defibrinated sheep blood (Thermo Fisher Scientific, USA) for 24 h at 37° C. in an atmosphere of 5% CO₂.

### Determination of Minimum Inhibitory and Minimum Bactericidal Concentrations

Minimum inhibitory concentrations (MICs) of compounds/extracts were determined using a broth microdilution technique, following the recommendations of the Clinical and Laboratory Standards Institute. Briefly, fresh colonies of each strain were used to prepare the respective inocula with an optical density at 600 nm (OD600) equal to 0.1 (approximately 1\hat{ }10⁸ CFU/mL). For all strains, cation-adjusted Mueller-Hinton broth (MHB2, Sigma-Aldrich, USA) was used, but in the case of S. pyogenes, MBH2 was previously supplemented with 2.5% lysed horse blood (Thermo Fisher Scientific, USA). In 96-well, U-bottom, microplates, each compound was serially diluted in the respective medium from stock solutions (10 mg/mL in DMSO) to achieve in-test concentrations ranging from 0.5 to 512 μg/mL. Wells were inoculated so that the final concentration in eachwas of 5×10⁵ CFU/mL. The microplates were incubated at 37° C. for 24 h. The MIC was defined as the lowest concentration of compound inhibiting the bacterial growth visible to the naked eye. The concentration of DMSO in the highest in-test concentration did not affect the bacterial growth of the tested strains.

The minimum bactericidal concentration (MBC) was determined by spreading 10 μL on MH agar (or blood agar in the case of S. pyogenes) from each well showing no visible growth, with further incubation for 24 h at 37° C.; the lowest concentration at which no growth occurred on MH plates was defined as the MBC.

### Biofilm Formation Inhibition Assay

Biofilm-associated infections remain a significant clinical challenge since the conventional antibiotic treatment or combination therapies are largely ineffective.

The capacity of all compounds/extracts to interfere with the biofilm formation by P. aeruginosa ATCC 27853 and S. aureus ATCC 29213 was assessed, using the crystal violet assay. TSB was used for P. aeruginosa ATCC 27853, whereas TSB supplemented with 1% glucose (Biochem Chemopharma, France) (TSBG) was used for S. aureus ATCC 29213. All compounds and extracts were tested at three concentrations (256, 64 and 16 μg/mL), which were necessarily sub-inhibitory concentrations (below the MIC). At least three independent experiments were performed in triplicate.

### EXAMPLES

### Example 1. Extraction of Anthocyanins

In the present invention, three anthocyanin extracts were obtained by extraction from elderberries, blackberries, and purification from a young red wine.

The Cy-3-glc was obtained by fractionation of a blackberry extract in a Buchner funnel system with a porous plaque (porosity 3) under vacuum using RP-18 gel as stationary phase and 10% (v/v) aqueous ethanol acidified with HCl as mobile phase. Posteriorly, the cy-3-glc fraction was further isolated through RP-18 gel column chromatography with an aqueous solution of 10% (v/v) ethanol acidified with HCl. The Mv-3-glc was obtained from a young red wine extract and their fractionation was similar to cy-3-glc being this pigment isolated with 20% (v/v) ethanol acidified with HCl. Also, complete elderberry and red wine extract anthocyanin extracts were used for the hemi-synthesis of pyranoanthocyanins.

### Example 2. Hemi-Synthesis of Pyranoanthocyanins

Non-glycosylated on position 5-O anthocyanins are subjected to a hemi-synthesis process.

### Pyranoanthocyanin Pigments

Four 3-O-glycosilated pyranoanthocyanin derivatives were synthesized. Carboxypyranoanthocyanins (Compounds II) were obtained from the reaction of cyanidin-3-O-glucoside and malvidin-3-O-glucoside with pyruvic acid using a molar ratio 1:100, at pH 2.6, during 11 days at room temperature and under stirring. Methylpyranoanthocyanins (Compounds III) were obtained from the reaction of cyanidin-3-O-glucoside and malvidin-3-O-glucoside with acetone using a 10% (v/v) aqueous solution of acetone at 37° C. during 7 days. For both reactions, the formation and purity of the 3-O-glycosilated pyranoanthocyanin derivatives were monitored by HPLC-DAD. The solvents were A (7.5% formic acid in water) and B (7.5% formic acid in acetonitrile) and the gradient used was 3 to 30% of solvent B, during 35 min at flow rate of 1.0 mL/min. The column was washed with 100% B during 6 min and stabilized with the initial conditions for more 9 min. After the maximum yield of reaction was obtained, the compounds formed were purified by RP-18 gel in a Büchner funnel system G3 and in column chromatography, eluted with an aqueous solution of 30% (v/v) methanol acidulated with HCl.

The bluish pigment pyranocyanidin-3-O-glucoside-butadienylidene-sinapyl (Compound VIII-B) was synthesised from the reaction of methylpyranocyanidin-3-O-glucoside (III-A) with sinapaldehyde. 5 mg of methylpyranocyanidin-3-glucoside were left to react at 37° C. with 21.43 mg of sinapaldehyde (molar ratio of 1:10) in 5 mL of an aqueous solution of 20% (v/v) ethanol at pH 4.0 (adjusted with a NaOH 0.01 M solution). The pyranoanthocyanins maximum yield was achieved after 10 days. The UV-Vis spectrum of this new compound recorded from the HPLC-DAD detector shows an absorption maximum wavelength of 563 nm. This compound was purified by TSK Toyopearl column chromatography eluting with an aqueous solution of 50% (v/v) methanol and isolated by semi-preparative HPLC for further structural characterization by NMR.

Vinylpyranomalvidin-3-O-glucoside-cathechin (compound VI-B) is obtained from the reaction of carboxypyranomalvidin-3-O— glucoside (II-B) with (+)-catechin (molar ratio 1:100) in the presence of acetaldehyde (molar ratio catechin/acetaldehyde 2:1), in 20% aqueous ethanol, pH 1.5 and at 37° C. The reaction is monitored by HPLC-DAD analysis. After 15 days, the reaction is stopped, and the major compounds formed is purified. The reaction mixture is applied directly onto a 250 mm×16 mm i.d. TSK Toyopearl gel HW-40(S) column and eluted with 80% aqueous methanol. The isolated pigment is then submitted to further purification which consisted in a final elution on silica gel 100 C18-reversed phase using a vacuum filtration system. The sample is applied on the top of a filtration funnel and the elution was firstly performed with deionized water in order to remove inorganic salts and any other organic impurities. The purified pigment is then recovered with 100% of methanol acidified with HCl. After methanol evaporation, the sample was frozen and lyophilized until further analysis.

A similar reaction is performed between the coumaroyl derivative of carboxypyranomalvidin-3-glucoside and (+)-catechin in the presence of acetaldehyde to get Vinylpyranomalvidin-3-O-cumaroylglucoside-cathechin (compound VI-A). The reaction conditions and all purification procedures are the same as already described.

Cyanidin-3-O-glucoside-4-vinylcatechol (Compound V-A) and malvidin-3-O-glucoside-4-vinylcatechol (Compound V-B) pigments can be synthesised from the reactions of 100 mg of cyanidin-3-O-glucoside or 100 mg of malvidin-3-O-glucoside with 372 mg and 341 mg of caffeic acid, respectively (molar ratio 1:10) in 100 mL of an aqueous solution of 20% (v/v) ethanol at room temperature. The formation of the new dyes was followed by HPLC-DAD, using the same method described for the others pyranoanthocyanin pigments. The maximum yield of the reaction products was reached after 17 days, and both reactions mixtures were purified through the RP-18 gel in a Buchner funnel system G3 and in a column chromatography, and both were eluted with an aqueous solution about 40% (v/v) methanol acidulated with HCl.

Pyranoanthocyanin dimer (Compound IX-A) is formed from the reaction of carboxypyranomalvidin-3-O-glucoside (II-B) (2 μM) with the methylpyranomalvidin-3-O-glucoside (III-B) (1.4 μM) in 40 mL of an aqueous solution of 20% ethanol (v/v), at pH 4.0 and at 37° C. The formation of new compound is monitored on a HPLC-DAD with the maximum formation of the new compound being reached after 14 days. Then, the reaction mixture is pre-purified by RP-18 gel in a Buchner funnel system G3 and the major compound formed is eluted with a 70% (v/v) methanol aqueous solution, acidulated with HCl.

### Amino-Based Pyranoanthocyanins

Pyranocyanidin-3-O-glucoside-(4-dimethylamino)-cinnamyl (Compound V-C) 100 mg of cyanidin-3-O-glucoside were left to react at 37° C. with 395 mg of 4-(dimethylamino)-cinnamic acid (molar ratio of 1:3) in 100 mL of an aqueous solution of 20% (v/v) ethanol at pH 3.2. The formation and purity of this new pigment was followed by HPLC-DAD, using a reverse-phase C18column 250×4.6 mm i.d., with particle size 5 μm. The solvents used were: A, 10% (v/v) formic acid in water and B, 0.5% (v/v) formic acid in 80% (v/v) acetonitrile and the gradient consisted in 20 to 85% of solvent B, during 70 min at flowrate of 1.0 mL/min. Then, the column was washed with 100% B during 10 min and stabilized with the initial conditions for more 10 min. After the compound reached its maximum formation yield, the reaction was stopped and the dye was purified by RP-18 gel in a Buchner funnel system G3 and in column chromatography, eluted with an aqueous solution of about 60% (v/v) methanol acidulated with HCl.

The synthesis of these two blue dyes 4-(Dimethylamino)-cinnamyl-10-vinylene-pyranocyanidin-3-O-glucoside (Compound VII-A) and 4-(Dimethylamino)-cinnamyl-10-vinylene-pyranomalvidin-3-O-glucoside (Compound VII-B) was performed through the reaction of carboxypyranocyanidin-3-O-glucoside
(II-A) and carboxypyranomalvidin-3-O-glucoside (II-B) (1 mol) with 4-(dimethylamino)-cinnamic acid (10 mol), in an aqueous solution of 20% (v/v) ethanol at pH 1.5 (adjusted with a 2% HCl solution) and at 37° C. during 7 days. The formation and purity of these new compounds was monitored by HPLC-DAD on a 250×4.6 mm i.d. reverse-phase C18 column, according to the procedure explain previously.

The elution of the pigment was performed with 70% of methanol in aqueous solution acidulated with HCl.

4-(Dimethylamino)-cinnamyl-10-butadienylidene-pyranocyanidin-3-O-glucoside (Compound VIII-A) pigment was obtained from the reaction of the 20 mg of methylpyranocyanidin-3-O-glucoside (Compound II-A) with 19.94 mg of 4-(dimethylamino)-cinnamaldehyde (molar ratio of 1:3) in 20 mL of ethanol. The formation and purity of this new dye was monitored by HPLC-DAD. The product formed was then purified by a RP-18 gel in a Buchner funnel system G3 and in a column chromatography and it was eluted with a solution of 70% (v/v) of methanol acidulated with HCl.

### Example 3. Pyranoanthocyanins

Pyranoanthocyanins prepared as described in Example 2 can be characterized according to formula I to IX of FIG. 1.

| | | | MW | λ | |
|---|---|---|---|---|---|
| Code | Name | Structure¹ | (g/mol) | (nm)² | SPF |
| II-A | Carboxypyrano- cyanidin-3-glucoside | 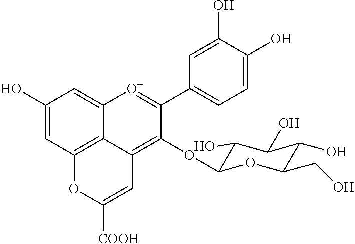 [SMILES: O=C(O)C1=Cc2c(OC3OC(CO)C(O)C(O)C3O)c(-c3ccc(O)c(O)c3)[o+]c3cc(O)cc(c23)O1] | 552.45 | 505 | 20.71 ± 0.18 |
| III-A | Methylpyranocyanidin- 3-glucoside | 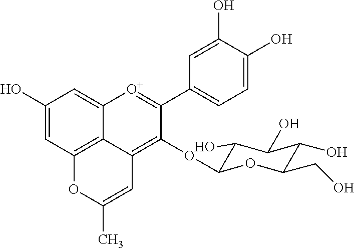 [SMILES: CC1=Cc2c(OC3OC(CO)C(O)C(O)C3O)c(-c3ccc(O)c(O)c3)[o+]c3cc(O)cc(c23)O1] | 522.45 | 473 | 18.53 ± 0.15 |
| V-C | Pyranocyanidin-3- glucoside-(4- dimethylamino)- cinnamyl | 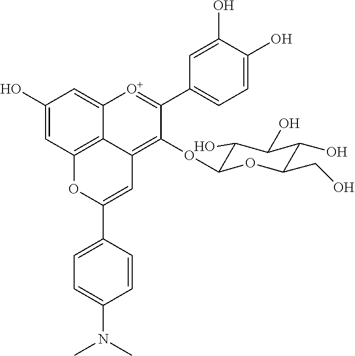 [SMILES: CN(C)c1ccc(C2=Cc3c(OC4OC(CO)C(O)C(O)C4O)c(-c4ccc(O)c(O)c4)[o+]c4cc(O)cc(c34)O2)cc1] | 627.45 | 560 | 13 ± 0.04 |
| VII-A | 4-(Dimethylamino)- cinnamyl-10-vinylene- pyranocyanidin-3- glucoside | 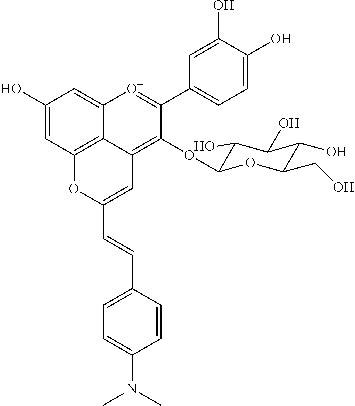 [SMILES: CN(C)c1ccc(/C=C/C2=Cc3c(OC4OC(CO)C(O)C(O)C4O)c(-c4ccc(O)c(O)c4)[o+]c4cc(O)cc(c34)O2)cc1] | 653.45 | 633 | — |
| VIII-A | 4-(Dimethylamino)- cinnamyl-10- butadienylidene- pyranocyanidin-3-O- glucoside | 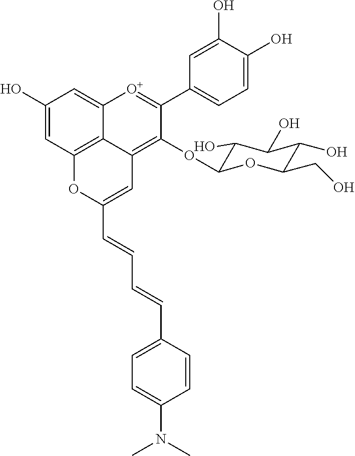 [SMILES: CN(C)c1ccc(/C=C/C=C/C2=Cc3c(OC4OC(CO)C(O)C(O)C4O)c(-c4ccc(O)c(O)c4)[o+]c4cc(O)cc(c34)O2)cc1] | 679.45 | 530 | 14.78 ± 0.02 |
| VIII-B | PyranoCyanidin-3-O- glucoside- butadienylidene- sinapyl | 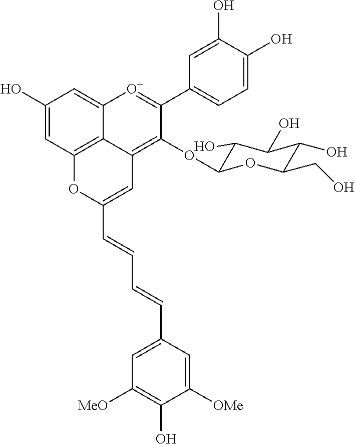 [SMILES: COc1cc(/C=C/C=C/C2=Cc3c(OC4OC(CO)C(O)C(O)C4O)c(-c4ccc(O)c(O)c4)[o+]c4cc(O)cc(c34)O2)cc(OC)c1O] | 712.45 | 563 | — |
| II-B | Carboxypyrano- malvidin- 3-glucoside | 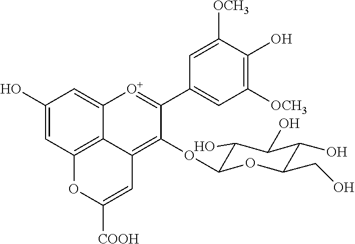 [SMILES: COc1cc(-c2[o+]c3cc(O)cc4c3c(c2OC2OC(CO)C(O)C(O)C2O)C=C(C(=O)O)O4)cc(OC)c1O] | 596.45 | 511 | 13.92 ± 0.04 |
| III-B | Methylpyrano- malvidin- 3-glucoside | 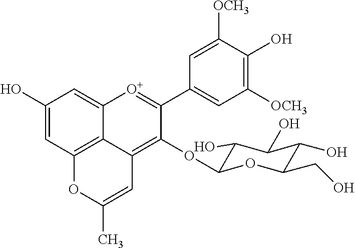 [SMILES: COc1cc(-c2[o+]c3cc(O)cc4c3c(c2OC2OC(CO)C(O)C(O)C2O)C=C(C)O4)cc(OC)c1O] | 566.45 | 478 | — |
| VI-A | Vinylpyrano- malvidin-3-O- cumaroylglucoside- cathechin | 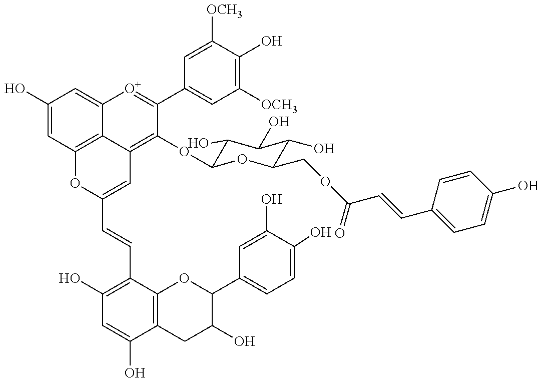 [SMILES: COc1cc(-c2[o+]c3cc(O)cc4c3c(c2OC2OC(COC(=O)/C=C/c3ccc(O)cc3)C(O)C(O)C2O)C=C(/C=C/c2c(O)cc(O)c3c2OC(c2ccc(O)c(O)c2)C(O)C3)O4)cc(OC)c1O] | 1012.45 | 577 | — |
| VI-B | Vinylpyranomal- vidin-3-O-glucoside- cathechin | 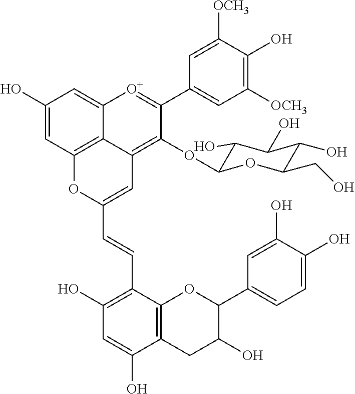 [SMILES: COc1cc(-c2[o+]c3cc(O)cc4c3c(c2OC2OC(CO)C(O)C(O)C2O)C=C(/C=C/c2c(O)cc(O)c3c2OC(c2ccc(O)c(O)c2)C(O)C3)O4)cc(OC)c1O] | 866.45 | 573 | 8.35 ± 0.06 |
| V-A | Cyanidin-3-O- glucoside-4- vinylcatechol | 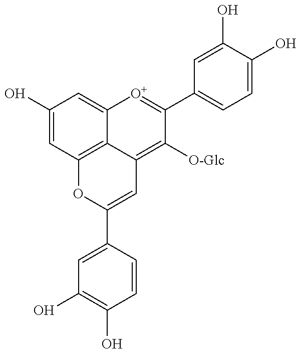 | 616.45 | 505 | 24.00 ± 0.01 |
| V-B | Malvidin-3-O- glucoside-4- vinylcatechol | 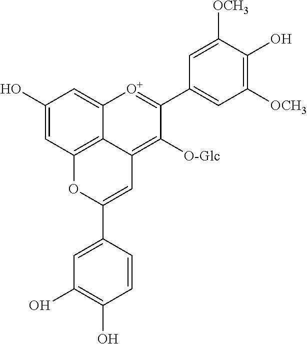 | 660.45 | 510 | 10.02 ± 0.00 |
| VII-B | 4-(Dimethylamino)- cinnamyl-10- vinylene- pyranomalvidin-3- glucoside | 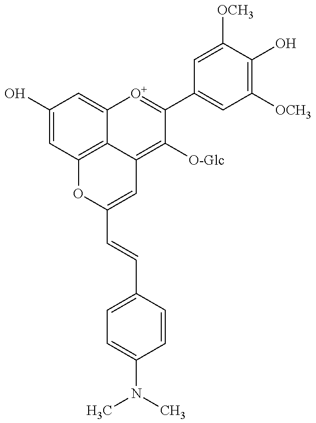 | 697.45 | 639 | 15.02 ± 0.07 |
| IX-A | Pyranomalvidin-3-O- glucoside-dimer | 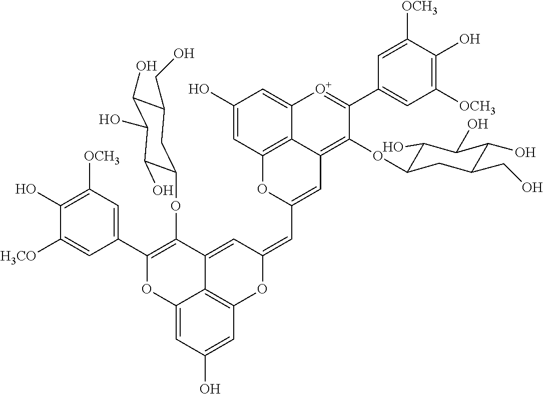 [SMILES: COc1cc(-c2oc3cc(O)cc4o/c(=C/C5=Cc6c(OC7CC(CO)C(O)C(O)C7O)c(-c7cc(OC)c(O)c(OC)c7)[o+]c7cc(O)cc(c67)O5)cc(c2OC2CC(CO)C(O)C(O)C2O)-c34)cc(OC)c1O] | 1080.45 | 674 | 19.99 ± 0.04 |

¹In all structures the counterion is Cl⁻.
²λ (nm: obtained from HPLC DAD

### Example 4. Cosmetic Formulations

| | | | Cytotoxicity |
|---|---|---|---|
| | | UV | (HaCat, HFF-1 |
| | Pyranoanthocyanin | protection | and Heka) |
| Formulation | composition | (0.2 mg/mL) | IC₅₀ (μM) |
| II-B | | 13.92 ± 0.04 | >100 |
| Extract | 31.55% of compound II-B | 21.60 ± 0.02 | — |
| II-B | 68.45% of compounds | | |
| | from family II | | |

None of the compounds or extracts tested exhibited antibacterial activity. Although a significant antibiofilm activity was observed (FIG. 11).

TABLE 1
Minimum inhibitory concentration (MIC) and minimum bactericidal concentration
(MBC) values (μg/mL) of extract II-B and compounds II-A, III-A and II-B against
P. aeruginosa ATCC 27853, S. aureus ATCC 29213, M. luteus ATCC 4698,
S. epidermidis ATCC 14990 and S. pyogenes ATCC 19615.

| | P. | S. | M. | S. | S. |
|---|---|---|---|---|---|
| | aeruginosa | aureus | luteus | epidermidis | pyogenes |
| | ATCC | ATCC | ATCC | ATCC | ATCC |
| Extract/ | 27853 | 29213 | 4698 | 14990 | 19615 |
| Compound | MIC (MBC) - μg/mL | | | | |
| Extract II-B | >512 (—) | >512 (—) | 512 (>512) | >512 (—) | >512 (—) |
| II-A | >512 (—) | >512 (—) | >512 (—) | >512 (—) | >512 (—) |
| III-A | >512 (—) | >512 (—) | >512 (—) | >512 (—) | >512 (—) |
| II-B | >512 (—) | >512 (—) | >512 (—) | >512 (—) | >512 (—) |

1. A process for extracting anthocyanins from foodstuffs or by-products, and to convert them into pyranoanthocyanins compounds characterized by comprising the following steps:
a) extracting anthocyanins with acidified hydroalcholic solutions;
b) converting the anthocyanins extracted from step (a) into pyranoanthocyanins compounds by subjecting non-glycosylated on position 5-0 anthocyanins to a hemi-synthesis process by reacting with a reagent to close ring A and C (R—C═CH₂, wherein R is a substituent that characterizes a family of compounds from I-IX) in aqueous solution at an approximate molar ratio compound/anthocyanin of 50:1.

2. A The process according to claim 1, wherein the foodstuffs or by-products are from winemaking industry and redberries.

3. A The process according to claim 1, wherein extracts comprising anthocyanins of step (a) are further concentrated by nanofiltration technologies and reduced to a fine powder by spray drying or lyophilization.

4. The process according to claim 1, wherein extracts are used in aqueous solution without being reduced to a fine powder.

5. A The process according to claim 3, wherein the fine powder comprising anthocyanins are further subjected to another extraction step by using hydroalcoholic solvents and purified by column chromatography with C18 reverse-phase gel by low-pressure column chromatography.

6. The process according to claim 1, wherein the extracts of step (b) are purified by reversed-phase silica gel column chromatography with the anthocyanin-pyruvic acid adducts fraction eluted with an alcoholic solution, preferably a water/ethanol solution.

7. A pyranoanthocyanin obtained by the process as described in claim 1, characterized by being represented by formulae I to IX 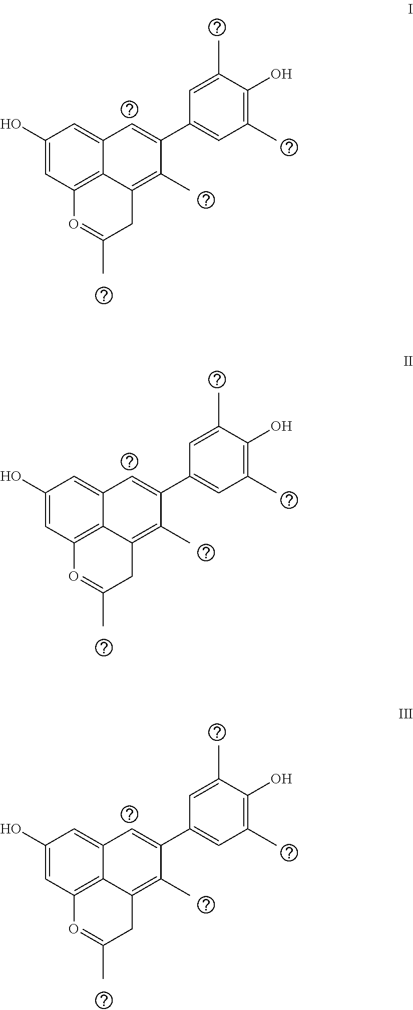 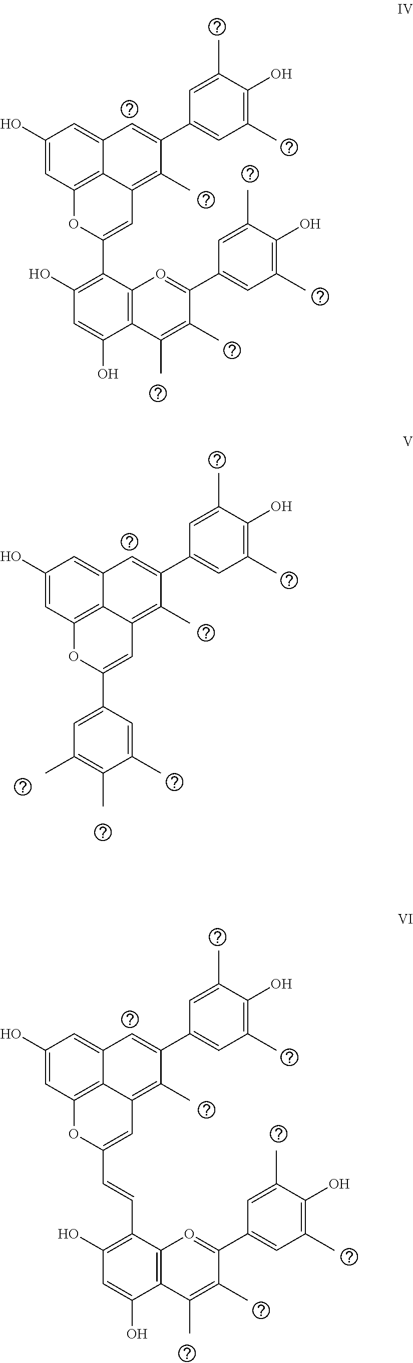 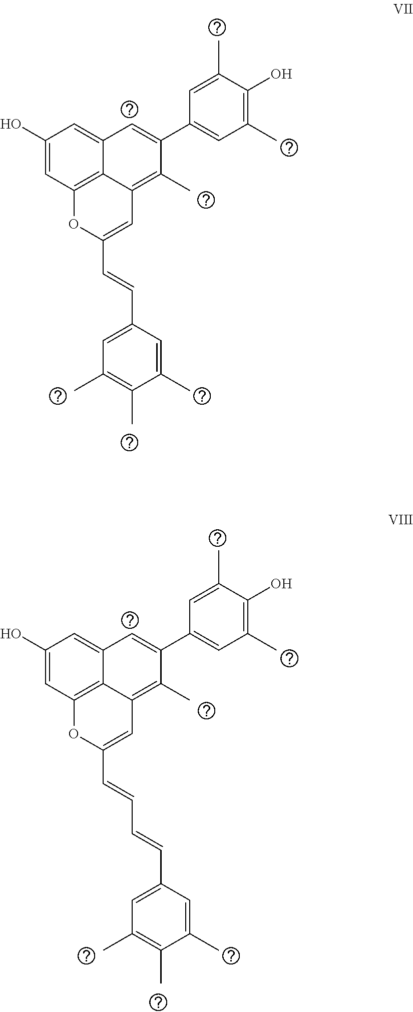 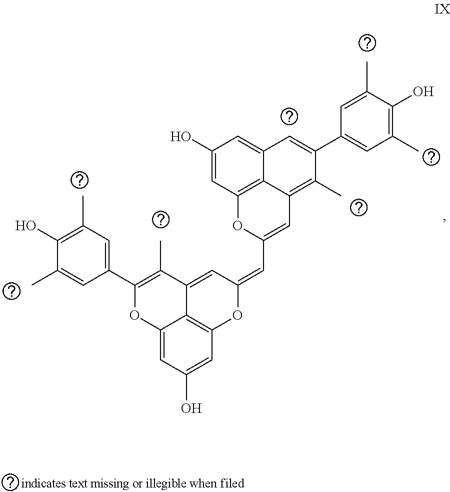
wherein:
R₁, R₂═H, OCH₃, OH
R₃═H, OH, sugar or acylated sugar
R₄=0, H, OH
R₅═H, OH, sugar
R₆═H, OH, OCH₃
R₇═H, OH, OCH₃, N(CH₃)₂
R₉═H, OH, OCH₃

8. A cosmetic formulation comprising the pyranoanthocyanins of formulae I to IX as described in claim 7 having a pH value in the range of 4 to 6.

9. The cosmetic formulation according to claim 8 comprising an amount of pyranoanthocyanins up to 0.5%.

10. The process according to claim 4, wherein the aqueous solutions comprising anthocyanins are further subjected to another extraction step by using hydroalcoholic solvents and purified by column chromatography with C18 reverse-phase gel by low-pressure column chromatography.
